(19) United States
(12) Patent Application Publication
(10) Pub. No.: US 2005/0009739 A1
(43) Pub. Date: Jan. 13, 2005

(54) Formulation strategies in stabilizing peptides in organic solvents and in dried states

(76) Inventors: Wei Wang, Alameda, CA (US); Yu-chang John Wang, Burlingame, CA (US); Sheryl Martin-Moe, Alameda, CA (US)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/500,680

(22) PCT Filed: Feb. 14, 2003
(86) PCT No.: PCT/US03/04790

Related U.S. Application Data
(60) Provisional application No. 60/356,915, filed on Feb. 14, 2002.

(51) Int. Cl. A61K 38/22

(52) U.S. Cl. 514/8; 514/6; 514/12

(57) ABSTRACT

The invention relates to stabilized formulations of therapeutically active peptides, particularly PACAP 66. Formulations of the invention include a peptide containing at least one histidine residue, a transition metal salt and an organic solvent. The above formulations may contain peptides that have at least one asparagine residue and are acidified and dried (such as spray-dried or freeze-dried) before formulation preparation. Other formulations of the invention relate to stabilized formulations of PACAP 66 or peptides containing an asparagine residue, which are acidified and dried (such as spray-died or freeze-dried) with or without a transition metal salt.

### FIELD OF THE INVENTION

The invention is generally related to the field of pharmaceutical formulations. More specifically, the invention is directed to stabilized formulations of therapeutically active peptides in an organic solvent, in an organic solvent-based suspension, or in a dried, such as lyophilized or spray-dried, state.

### BACKGROUND OF THE INVENTION

Therapeutic peptides are susceptible to aggregation and/or chemical degradation when stored in an aqueous solution for extended periods of time. This tendency of peptides to aggregate or degrade is generally characterized as “instability” and may be measured by many different analytical methods, such as UV/VIS spectrophotometry, Reversed Phase High Performance Liquid Chromatography (RP-HPLC), Capillary Electrophoresis (CE), etc. The instability of peptides in an aqueous solution may be minimized by a variety of strategies. Wang, Int. J. Pharm., 85:129-88 (1999); Arakawa, et al., Adv. Drug Deliv. Rev. 46:307-26 (2001). Two often-used strategies are to formulate the peptides with a proper amount of a stabilizer(s) or to dry (such as spray-dry, freeze-dry) the peptide for long-term storage.

A rare method of stabilizing peptide for long-term storage is mixing the peptide with a non-aqueous organic solvent. Organic solvents may improve the stability of peptides by promoting formation of secondary structures (Zou and Sugimoto, Biometals, 13:349-59 (2000); Kozin, et al., Biochem. Biophys. Res. Commun., 258:959-64 (2001)) and by inhibiting certain chemical reactions, such as hydrolysis (Brennan and Clarke, Protein Sci., 2:331-38 (1993)). Peptide deamidation can be modestly inhibited in an aqueous solution upon addition of an organic solvent, such as glycerol (Li, et al., J. Pept. Res. 56:326-34 (2000)), and ethanol or dioxane (Brennan and Clarke, supra). For example, the stability of leuprolide, a 9-amino acid peptide hormone, has an overall better stability in dimethyl sulfoxide (DMSO) than in water. Hall, et al., J. Pept. Res., 53:432-41 (1999); Stevenson, et al., Int. J. Pharm., 191:115-29 (1999).

The native pituitary adenylate cyclase-activating polypeptide (PACAP) is a peptide hormone with less than 40 amino acids. Vaudry, et al., Pharmacol. Rev., 52:269-324 (2000). Based on its sequence, PACAP is a member of a superfamily of peptide hormones, including vasoactive intestinal peptide (VIP), glucagon, growth hormone releasing factor (GRF), and secretin (Vaudry, et al., supra). By binding to different receptors, PACAP initiates a variety of pharmacological activities, one of which is the stimulation of insulin secretion. As discussed in a related application (co-owned, co-pending U.S. Ser. No. 09/671,773, WO 01/23420), PACAP without modification is not suitable to treat type II diabetes, because significant side effects may occur. In search of a PACAP-like peptide(s) that can be used safely to treat type II diabetes, a variety of PACAP analogues were synthesized and PACAP 66 was identified. PACAP 66 is the same molecule as “R3P 66” which is disclosed in U.S. Ser. No. 09/671,773 and in WO 01/23420, both of which are incorporated herein by reference. The peptide sequence for PACAP 66 is HSDAVFTDNYTRLRKQVAAKKYLQSIKNKRY (SEQ ID NO: 1).

The degree of instability of PACAP 66 has, however, been found to be far greater than what is expected of a peptide in general. In the evaluation of its stability, we found that PACAP 66 was not stable enough in an aqueous environment. Furthermore, addition of a potential formulation stabilizer did not improve its stability. Among the excipients we tested were different metal ions, such as zinc, magnesium, or calcium, but none of these ions improved the stability of the peptide. (See FIG. 1.)

In order to overcome this stability barrier and, in turn, increase the product shelf life, preparations of the peptide in organic solvents were made. Fortunately, the peptide dissolved easily in some organic solvents, but, surprisingly, the stability of this peptide in these organic solvents was as poor as, or even worse than, in an aqueous environment. (See FIGS. 1 and 2 for comparison.) Many potential peptide stabilizers in an aqueous solution, such as sugars, are not readily soluble in organic solvents, and therefore could not be used. Many other known strategies for peptide stabilization were tried without success. New methods and formulations for the stabilization of PACAP 66 were therefore needed. Such methods yielded novel formulations and methods that are extendible to other peptides. The present invention therefore provides novel methods of controlling peptide instability in organic solvents, in organic solvent-based suspensions, and in dried states.

### SUMMARY OF THE INVENTION

The invention provides formulations of peptides either in suspension or solution, or freeze- or spray-dried, that are stabilized by a transition metal salt, an acid or both. In an embodiment of the invention, formulations, either in suspension or solution or dried, include a peptide containing at least one histidine residue and a transition metal salt. The transition metal salt may be a salt of a transition metal selected from zinc, copper, iron, manganese, nickel or cobalt, and is preferably zinc. The histidine residue of the peptide may be a terminal histidine residue. The peptide is preferably PACAP 66, but may include other peptides, such as, for example, PACAP, PACAP-like peptides, VIP, glucagon, glucagon-like peptides, GRF, secretin, helodermin, exendin-4, and functionally equivalent variants thereof. Also included may be adrenocorticotropic hormone, angiotensins, renin substrate tetradecapeptide, natriuretic peptides, gastrointestinal peptides, luteinizing hormone releasing hormone, melanocyte sitmulating hormone, and neurotensin, and parathyroid hormone.

In another embodiment, such formulations of the invention include an organic solvent. The organic solvent may be; for example, DMSO, 1-methyl-2-pyrrolinidone, propanol, propylene glycol, glycerol acetate, monothioglycerol, acetic acid, diethanolamine, benzyl alcohol, ethyl lactate, glycerol formal, N-methylpyrrolidone, polyethyleneglycol 400, and isopropyl myristate, or may be a mixture of two or more of these solvents. The organic solvent is preferably DMSO, 1-methyl-2-pyrrolinidone or propanol. In one embodiment of the invention, the molar ratio of zinc salt to peptide in the organic solvent is above 0.1.

In another embodiment of the invention, formulations of the invention include dried formulations containing a peptide having at least one asparagine residue and an acid. The acid may be TFA or is an inorganic acid, such as, for example, HCl and H₃PO₄. Such formulations may be spray- or freeze-dried. Such formulations may also contain a transition metal salt, as described above. In one embodiment of this formulation, the peptide is PACAP 66 and/or a salt thereof Finally, such formulations may also contain an organic solvent, as described above.

The invention also relates to processes for manufacturing the formulations detailed above. Such processes include preparing an acid solution in water, cooling the acid solution to below room temperature, mixing the cooled solution with a peptide containing at least one asparagine residue, as described above, and then drying the resulting mixture, preferably by spray- or freeze-drying. A transition metal salt, as described above, may be added to the cooled solution before drying. The acids and peptides for use in processes of the invention are as described above.

In another process of the invention, a transition metal salt, as described above, is mixed with a peptide containing at least one histidine residue, as described above, and then dried, preferably by spray- or freeze-drying. An organic solvent, as described above, may also be added to the mixture.

The invention is described in more detail below by the following drawings, description and claims.

### BRIEF DESCRIPTION OF THE DRAWINGS

FIG. 1 shows the stability of PACAP 66 in an aqueous solution at 40° C. in the presence of different metal ions.

FIG. 2A shows the stability of PACAP 66 in DMSO at 40° C. in the presence of different metal ions as analyzed by RP-HPLC.

FIG. 2B shows the stability of PACAP 66 in DMSO at 40° C. in the presence of different metal ions as analyzed by CE.

FIG. 3 shows the stability of acidified, lyophilized PACAP 66 in DMSO at 40° C.

FIG. 4 shows the effect on the stability of PACAP 66 in DMSO at 40° C. of HCl or a combination of HCl and ZnCl₂.

FIG. 5 shows the effect on the stability of PACAP 66 in 1-methyl-2-pyrrolinidone at 40° C. of HCl or a combination of HCl and ZnCl₂.

FIG. 6 shows the effect on the stability of PACAP 66 in 2-propanol at 40° C. of ZnCl₂.

FIG. 7 shows the effect on the stability of lyophilized PACAP 66 at 40° C. of HCl or a combination of HCl and ZnCl₂.

FIG. 8A shows an NMR spectrum of PACAP 66 in DMSO in the absence of ZnCl₂.

FIG. 8B shows an NMR spectrum of PACAP 66 in DMSO in the presence of ZnCl₂.

### DETAILED DESCRIPTION OF THE INVENTION

The invention relates to stabilized peptide formulations. Peptide formulations of the invention include organic, anhydrous solutions, suspensions, or dried solids, which are stabilized by addition of a metal ion, by acidification and drying of the peptide, or by a combination of the two methods. Specific embodiments of the invention include stabilized formulations of PACAP 66, or “R3P 66” (SEQ ID NO: 1).

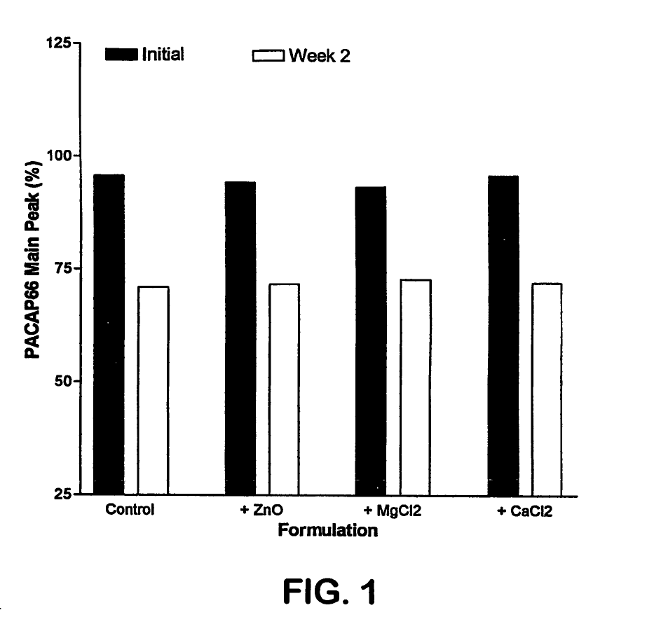

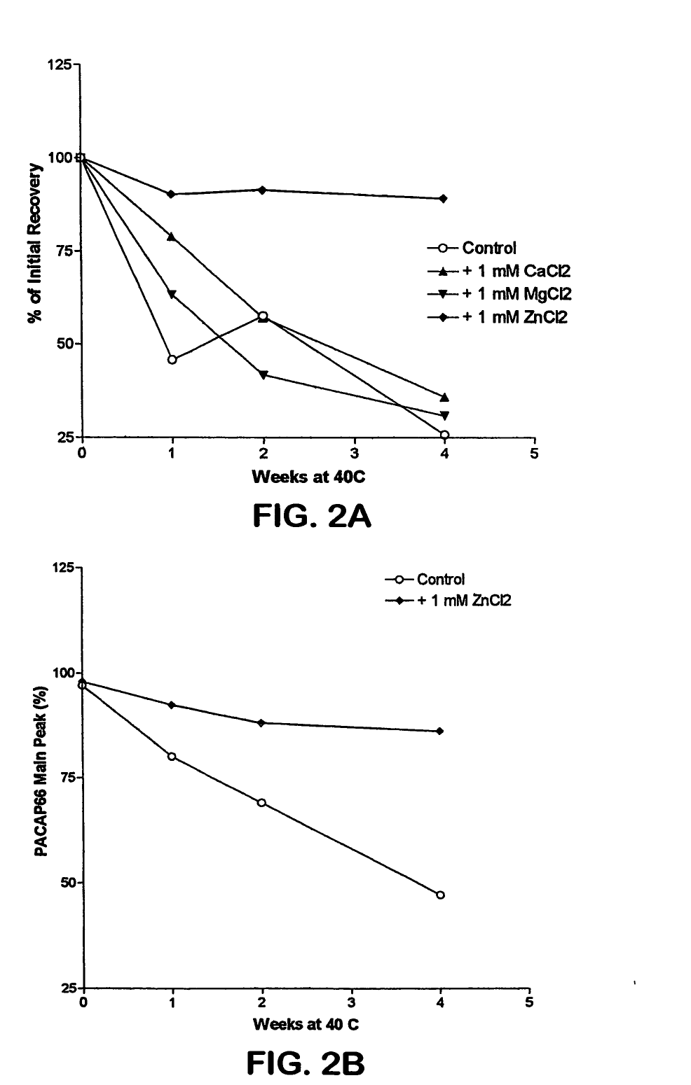

PACAP 66 is not stable in an aqueous environment Addition of different metals, such as zinc, magnesium, or calcium, does not improve its stability (FIG. 1). This appears to be caused by peptide autolysis, as was seen with VIP, a closely related peptide. Mody, et al., Int. J. Pept. Protein Res., 44, 441 447 (1994). In pursuing methods of stabilizing PACAP 66, we evaluated the stability of this peptide in organic solvents. We initially found that the stability of this peptide in several organic solvents was unsatisfactory, or even worse than that observed in an aqueous environment. (See FIGS. 1 and 2 for a comparison.)

To improve the stability of PACAP 66 in these organic solvents, we designed a variety of stabilizing strategies, and a few of these proved to be unexpectedly effective. These include two approaches that turned out to be very effective in stabilizing the peptide: (1) addition of a metal salt, such as, for example, zinc chloride, in an organic solvent and (2) acidification of the peptide in an aqueous solution followed by drying. The stabilization of PACAP 66 in an organic solvent by zinc salt was surprising, because several metal salts failed to stabilize PACAP 66 in an aqueous solution. (See, e.g., FIG. 1). It was also surprising to find that the peptide was much more stable in an organic solvent after the peptide was acidified and dried, because acidification of a peptide solution usually leads to increased hydrolysis of the peptide. These stabilization strategies were also found to be effective in organic solvent-based suspensions and in a dried state during storage. In the following section, these successful strategies and the stabilization mechanisms that made them successful are more fully described. The implications of these findings and possible medical uses of the formulations are also described below.

### Strategies in Stabilizing Peptides in Organic Solvents

(1) Use of Specific Metal Ions

Different metal salts, including ZnCl₂, MgCl₂, and CaCl₂ were separately dissolved at 1 mM in DMSO, a non-aqueous organic solvent. PACAP 66 was then dissolved in these solutions at 2 mg/mL. The bulk solution was aliquoted into 2-mL screw-capped (with an o-ring) sterile polypropylene vials. These stability samples were incubated at 40° C. and analyzed at predetermined intervals.

FIG. 2 shows the stability of PACAP 66 in DMSO as determined by the peptide recovery (RP-HPLC) and purity (CE) at 40° C. in the presence of different metal salts. More than 70% of PACAP 66 was degraded in DMSO in 4 weeks at 40° C. by RP-HPLC, but only approximately 10% of PACAP 66 was degraded in the presence of 1 mM ZnCl₂ under the same storage conditions. The other samples, containing MgCl₂, and CaCl₂ did not have any significant stabilizing effect when compared with the control. The CE results were similar to the findings from the RP-HPLC analysis. The purity of PACAP 66 in the 4-week control stability sample by CE was higher than the recovery by RP-HPLC, suggesting that certain PACAP 66 degradation products might have a different UV response or were not well separated from the main peak by CE.

(2) Acidification and Lyophilization of PACAP 66

Several acid solutions were prepared at 0.1%, including HCl, trifluoro acetic acid (TFA), and H₃PO₄ and cooled to 2-8° C. The cold acid solutions were then mixed with PACAP 66 at a PACAP 66:acid molar ratio of 1:10. After mixing, the cold PACAP 66 solutions were immediately placed inside a precooled freeze-drier and were lyophilized. The lyophilized material was further equilibrated in a desiccator containing P₂O₅ for at least one day to absorb additional moisture from the lyophilized peptide. The acidified and dried material was then dissolved in DMSO at 2 mg/mL and stability was conducted as described in the upper section.

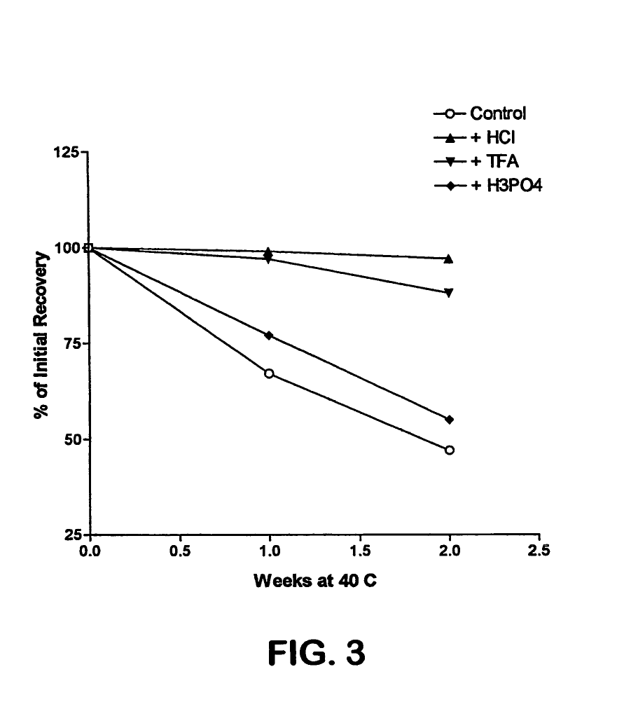

FIG. 3 shows the stability of acidified and lyophilized PACAP 66 in DMSO. More than 50% of unprocessed PACAP 66 was degraded in the control sample after storage at 40° C. for 2 weeks, while a lower percentage of degradation, less than 10%, was observed for samples containing acidified and lyophilized PACAP 66. The relative stabilization effect by these acids was HCl >TFA >H₃PO₄, in an apparent order of decreasing acidity. The recovery of HCl-acidified PACAP 66 at the end of a 2-week period was 97% by RP-HPLC. However, the recovery could be slightly overestimated, as the corresponding purity of PACAP 66 in the sample was only 89% by RP-HPLC.

We understand that peptides can be hydrolyzed readily under acidic conditions in an aqueous solution. Secretin, a PACAP-like peptide, can be degraded easily in an aqueous solution at pH 4. In the acidification of PACAP 66, the pH of acidified PACAP 66 solution was measured to be 2.2 after addition of TFA. At this pH, PACAP 66 should be rapidly hydrolyzed. However, the acidification process was conducted at a low temperature, followed by immediate lyophilization, and no detectable hydrolysis in PACAP 66 was observed.

In the investigation of the degradation mechanisms of PACAP 66 in DMSO, we found that the major degradation pathway of this peptide was dimerization. The peptide dimer was formed via a cyclic imide intermediate on the asparagine residues in the peptide. Severs, et al., “Instability of Asparagine and Aspartic Acid of a Polypeptide in DMSO”, WCBP,7th Symposium on the Interface of Regulatory and Analytical Sciences for Biotechnology Health Products, San Francisco, Calif. (2003). Therefore, acidification of the peptide inhibited dimerization through these amino acid residues in DMSO. (See also Mechanisms of PACAP 66 Stabilization, infra).

(3) Stabilization of PACAP 66 at High Concentrations, in Other Organic Solvents, in Organic Solvent Suspensions, and in Lyophilized State

To test whether a metal salt would stabilize PACAP 66 at a high peptide concentration in an organic solvent, a high concentration of a metal salt would be required, assuming a fixed ratio of metal and peptide is needed for stabilization. A metal salt, however, has limited solubility in an organic solvent Therefore, a similar preparation method was adopted for sample preparation of peptide-metal mixtures at high concentrations, as described under Acidification and Lyophilization of PACAP 66, supra. Briefly, a metal salt and the peptide were first dissolved at a fixed molar ratio in an aqueous solution. The solution was then aliquoted in 3-mL glass vials at a fixed volume and lyophilized. Stability samples were prepared by adding a fixed amount of an organic solvent in the vial. The sample vials were then capped, sealed, and incubated at 40° C. Stability samples were first diluted to a reasonable concentration before analysis by RP-HPLC or CE. Similarly, a peptide suspension was prepared by mixing a proper amount of an organic solvent in a sample vial containing the lyophilized mixture and incubated at 40° C. Stability of solid PACAP 66 was evaluated directly by incubating the sample vial containing the lyophilized mixture at 40° C.

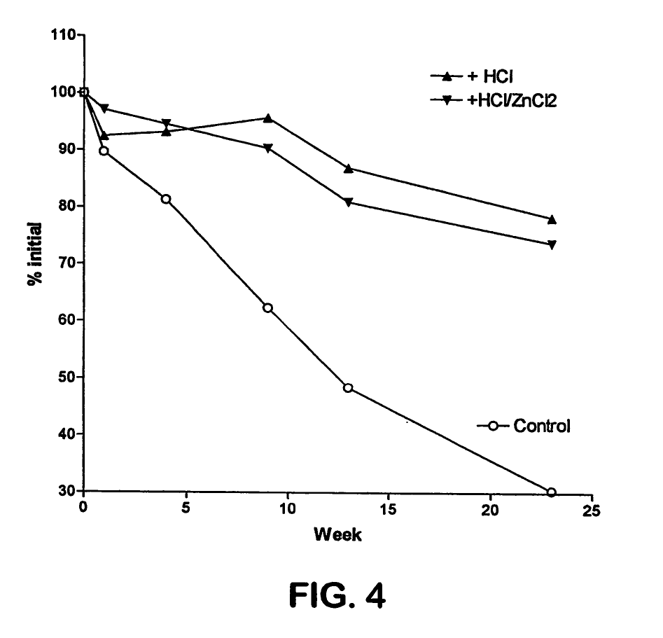

FIG. 4 shows the stability of PACAP 66 solution at 300 mg/mL in DMSO at 40° C. PACAP 66 in the sample was acidified in the absence and presence of ZnCl₂. Approximately 70% of the peptide was degraded in the control sample after storage for 23 weeks, while approximately 20% was degraded in the acidified samples in the presence or absence of ZnCl₂.

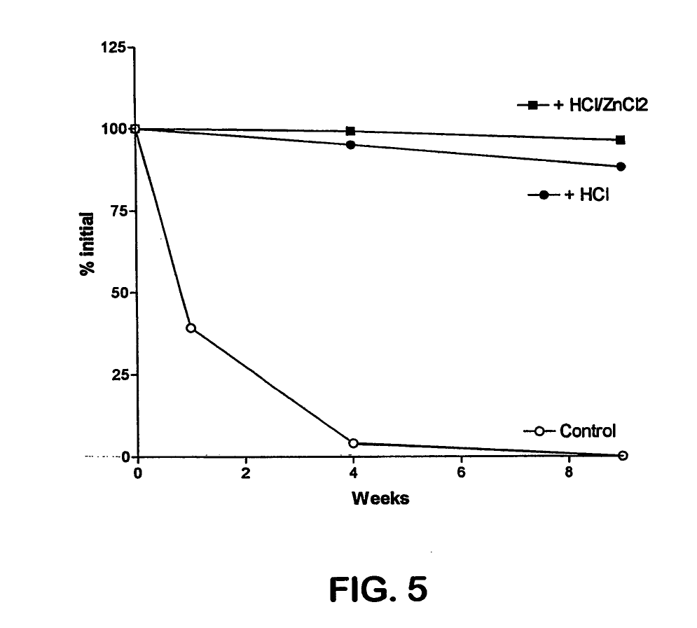

FIG. 5 shows the stability of PACAP 66 solution at 20 mg/mL in 1-methyl-2-pyrrolinidone at 40° C. PACAP 66 in the sample was acidified in the absence and presence of ZnCl₂. The peptide was degraded to a non-detectable level in the control sample after storage for 9 weeks, while more than 80% of the peptide remained in the acidified samples. Addition of ZnCl₂ seems to stabilize PACAP 66 to a higher degree.

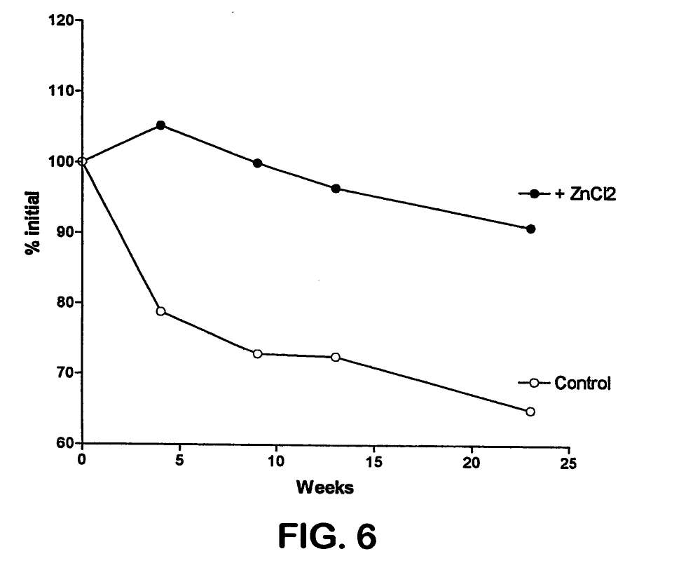

FIG. 6 shows the stability of PACAP 66 suspension at 20 mg/mL in 2-propanol at 40° C. Addition of ZnCl₂ significantly improved the storage stability of PACAP 66.

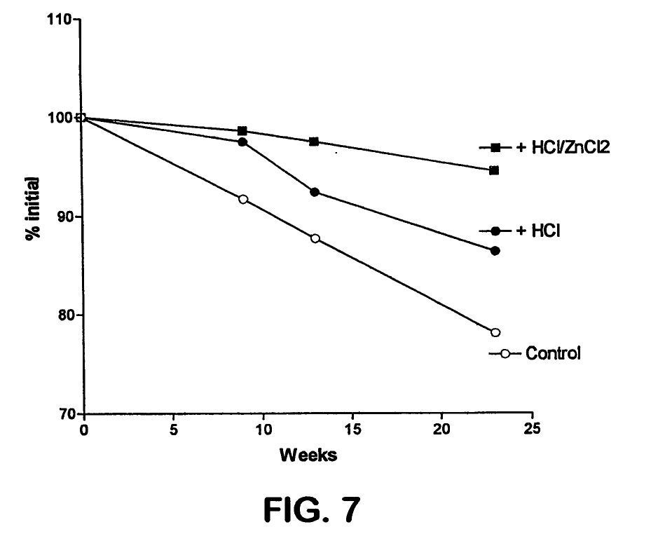

FIG. 7 shows the stability of PACAP 66 in a lyophilized state at 40° C. Acidification significantly stabilized the peptide during storage. Addition of ZnCl₂ seems to stabilize the peptide to a higher degree.

### Mechanisms of PACAP 66 Stabilization

(1) Metal Ion-induced PA CAP 66 Stabilization

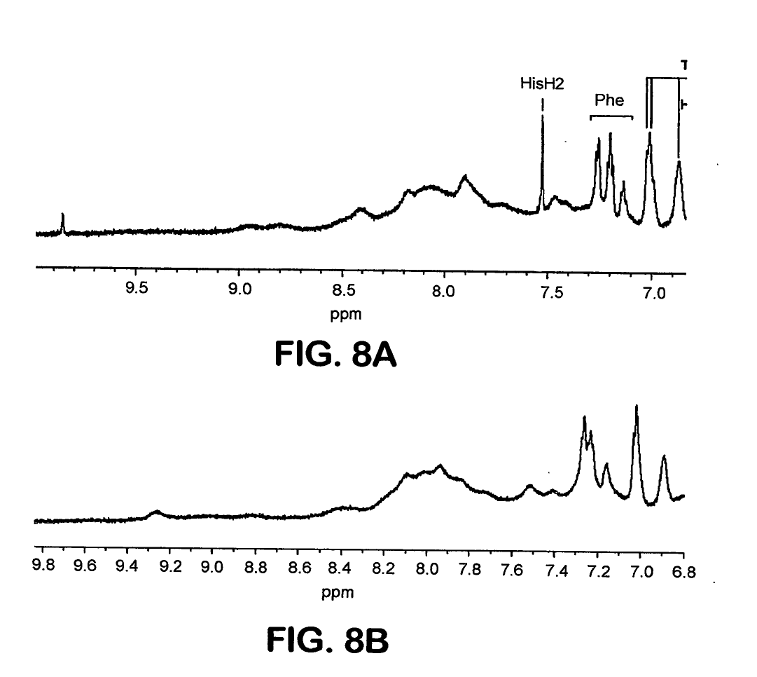

The results show that ZnCl₂ stabilized PACAP 66 in DMSO, while MgCl₂ and CaCl₂ did not This suggests that metal ions do not stabilize PACAP 66 simply by ionic interactions. Therefore, we proposed that zinc and PACAP 66 form a chelate complex via the N-terminal histidine residue, which hinders its own degradation. To prove our hypothesis, we measured the NMR spectrum of PACAP 66 in DMSO in the absence and presence of 1 mM ZnCl₂ (FIG. 8). The most dramatic difference in the spectrum in the presence of 1 mM ZnCl₂ is the disappearance of the histidine H2 and H4 signals in the broad amide background. This clearly suggests an interaction Of ZnCl₂ with the terminal histidine residue. On the other hand, the spectrum of PACAP 66 in D₂O is essentially the same in the absence or presence of ZnCl₂ (data not shown). Therefore, these results indicate that peptide-Zn interaction is present only in an organic solvent, not in an aqueous solution, and explain why zinc oxide at 10 mM did not stabilize PACAP 66 in an aqueous solution (FIG. 1).

The above conclusion on the mechanism of Zn-induced peptide stabilization is supported by data from several references. First, the formation of a metal-peptide complex was observed in PACAP-related peptides. One study showed that several PACAP fragments could form a complex with copper (II) in an aqueous solution. Kowalik-Jankcowska, et al., J. lnorg. Biochem., 76:63-70 (1999). One of these fragments is HSDGI-NH₂ and the first three amino acids (HSD) corresponds to the N-terminal sequence of PACAP 66. This PACAP fragment forms a dimeric complex (Cu₂-L₂) between pH 5 to 8 and monomeric complex (Cu-L) above pH 8 with a binding ratio of 1:1. It was shown that the third aspartic acid residue dramatically stabilized the complex. Although these copper complexes were identified, it was not mentioned whether the complex would enhance or compromise the stability of these peptide fragments. Second, zinc is able to form a complex with histidine residues in peptides, resulting in an altered stability behavior. Zn²⁺ has been shown specifically to interact with His13 and His14 in amyloid β-peptide, and the interaction altered the secondary structure of the peptide and its aggregation behavior. Yang, et al., Eur. J. Biochem., 267:6692-98 (2000). A more recent study showed that binding of Zn²⁺ to amyloid β-peptide(1-16) at a 1:1and 1:2 ratio (peptide/zinc) caused a change (more ordered) in secondary structure, leading to a more stable complex. Kozin, et al., supra. Again, the chemical stability of the peptide could not be predicted. Third, the second residue in PACAP 66 is serine, which has been shown to participate in formation of a zinc-peptide complex (Cung, et al., J. Biol. Chem., 263:5574-80 (1988)), and finally, the formation of a zinc-peptide complex may rigidify the peptide, affecting its stability. Haran, et al., Int. J. Pept. Protein Res., 20:380-86 (1982).

(2) Acidification-induced PACAP 66 Stabilization

As we discussed before, the major degradation pathway in PACAP 66 in DMSO is dimerization via the formation of a cyclic imide intermediate. It is well known tha the formation of the cyclic imide begins with the intramolecular, nucleophilic attack of the backbone nitrogen on the carbonyl group of the asparagine side chains. The formation of the cyclic imide is generally accelerated under a basic condition, as a basic condition favors deprotontation of the backbone nitrogen and the deprotonated nitrogen has a higher nucleophilicity. One the contrary, acidification of the peptide would favor protonation of the backbone nitrogen and slow down the reaction. At the same time, acidification generally facilitates peptide hydrolysis. This was not the case for PACAP 66, however, because the peptide was in a non-aqueous solution, suspension, or dried state.

### Implications of the Current Findings

For the first time, we demonstrated that ZnCl₂ can be used as a formulation excipient to stabilize a peptide in an organic solvent, in an organic solvent-based suspension, or in a dried state. Since PACAP 66, based on its sequence analysis, is a member of a superfamily of peptide hormones, it is anticipated that ZnCl₂ would stabilize any member of this superfamily in DMSO because of their structural similarities. These member peptides include vasoactive intestinal peptide (VIP), glucagon, glucagon-like peptides, growth hormone releasing factor (GRF), secretin, helodermin, and exendin-4. Based upon our analysis of the stabilization mechanisms, ZnCl₂ will also stabilize any peptide dissolved in DMSO which contains at least one histidine residue, such as adrenocorticotropic hormone, angiotensins, renin substrate tetradecapeptide, natriuretic peptides, gastrointestinal peptides, luteinizing hormone releasing hormone, melanocyte sitmulating hormone, and neurotensin, and parathyroid hormone.

Since zinc plays a clear role in the conformational integrity of insulin in the hexameric form and during storage of insulin in an aqueous solution or suspension, it is probable that zinc will stabilize insulin and other structurally dissimilar polypeptides in an organic solvent, in a solvent mixture, in an organic solvent-based suspension, or in a dried state.

It has been observed that several PACAP fragments could form a complex with copper (II) in an aqueous solution. This suggests that other transition metal ions, in addition to zinc may stabilize PACAP 66 in an organic solvent, in a solvent mixture, in an organic solvent-based suspension, or in a dried state. These transition metal ions may include, but are not limited to, copper, iron, manganese, nickel, and cobalt. Interaction and stabilization by these metals may also be applicable to other similar or dissimilar peptides, as discussed above.

In this application, we demonstrated stabilization of PACAP 66 at different concentrations in two different organic solvents by metal ions. It is very likely that zinc- or other metal-induced stabilization of PACAP 66, as well as similar or dissimilar peptides, is also operable in other organic solvents or solvent mixtures, including propylene glycol, s glycerol acetate, monothioglycerol, acetic acid, diethanolamine, benzyl alcohol, ethyl lactate, glycerol formal, N-methylpyrrolidone, polyethyeneglycol 400, isopropyl myristate and other alcohols.

In this application, we also demonstrated stabilization of PACAP 66 by acidification or by combination of acidification and use of metal ions in an organic solvent, in an organic solvent-based suspension, or in a dried state. It is conceivable that PACAP 66 or other peptides (aforementioned) are stabilized by the same strategies in different organic solvents (aforementioned), in different solvent mixtures, in suspensions of other organic solvents, and in a dried state. The dried peptide may be a mixture with any other formulation excipients, delivery vehicles, or other necessary components. Since acidification stabilized asparagine residues in PACAP 66, it is conceivable that other peptides containing asparagine residues are stabilized by acidification in an organic solvent, in an organic solvent mixture, in an organic solvent-based suspension, or in a dried state.

### Methods of Use

Formulations of the invention may be used to treat a variety of diseases and conditions depending on the nature and role of the peptide stabilized. Stabilized formulations of PACAP 66, particularly, maybe used in the treatment of diabetes and related conditions. Formulations of PACAP 66 may be used alone or in combination with other known diabetes treatments. Furthermore, formulations of PACAP 66 may be used in combination with other therapies to treat diseases or conditions often occurring in conjunction with diabetes and related disorders, such as obesity, lipid disorders and/or hypertension.

The dosage regimen to prevent, treat, give relief from, or ameliorate a diabetic condition or disorder, or to otherwise protect against or treat a diabetic condition with the combinations and formulations of the present invention is selected in accordance with a variety of factors. These factors include, but are not limited to, the type, age, weight, sex, diet, and medical condition of the subject, the severity of the disease, the route of administration, pharmacological considerations such as the activity, efficacy, pharmacokinetics and toxicology profiles of the particular inhibitors employed, whether a drug delivery system is utilized, and whether the formulations are administered with other active ingredients. Thus, the dosage regimen actually employed may vary widely and therefore deviate from the preferred dosage regimen set forth herein.

The total daily dose of each drug can be administered to the patient in a single dose, or in multiple subdoses. Typically, subdoses can be administered two to six times per day, preferably two to four times per day, and even more preferably two to three times per day. Doses can be in immediate release form or sustained release form sufficiently effective to obtain the desired control over the diabetic condition.

Formulations of the invention containing PACAP 66 may be used to treat diseases, such as diabetes, including Type 2 diabetes. Such methods may also delay the onset of diabetes and diabetic complications. Other diseases and conditions that may be treated or prevented using formulations of the invention include: Maturity-Onset Diabetes of the Young (MODY) (Herman, et al., Diabetes 43:40 (1994)), Latent Autoimmune Diabetes Adult (LADA) (Zimmet, et al., Diabetes Med. 11:299 (1994)), impaired glucose tolerance (IGT) (Expert Committee on Classification of Diabetes Mellitus, Diabetes Care 22 (Supp. 1) S5 (1999)), impaired fasting glucose (IFG) (Charles, et al., Diabetes 40:796 (1991)), gestational diabetes (Metzger, Diabetes, 40:197 (1991), and metabolic syndrome X.

Formulations of the invention containing PACAP 66 may also be used to treat secondary causes of diabetes (Expert Committee on Classification of Diabetes Mellitus, Diabetes Care 22 (Supp. 1), S5 (1999)). Such secondary causes include glucocorticoid excess, growth hormone excess, pheochromocytoma, and drug-induced diabetes. Drugs that may induce diabetes include, but are not limited to, pyriminil, nicotinic acid, glucocorticoids, phenytoin, thyroid hormone, β-adrenergic agents, α-interferon and drugs used to treat HIV infection.

The formulations of the invention containing PACAP 66 may be used alone or in combination with additional therapies and/or compounds known to those skilled in the art in the treatment of diabetes and related disorders. Alternatively, the formulations described herein may be used, partially or completely, in combination therapy.

The formulations of the invention containing PACAP 66 may also be administered in combination with other known therapies for the treatment of diabetes, including PPAR agonists, sulfonylurea drugs, non-sulfonylurea secretagogues, α-glucosidase inhibitors, insulin sensitizers, insulin secretagogues, hepatic glucose output lowering compounds, insulin and anti-obesity drugs. Such therapies may be administered prior to, concurrently with or following administration of the formulations of the invention containing PACAP 66. Insulin includes both long and short acting forms and formulations of insulin. PPAR agonist may include agonists of any of the PPAR subunits or combinations thereof. For example, PPAR agonist may inlcude agonists of PPAR-α, PPAR-γ, PPAR-δ or any combination of two or three of the subunits of PPAR. PPAR agonists include, for example, rosiglitazone and pioglitazone. Sulfonylurea drugs include, for example, glyburide, glimepiride, chlorpropamide, and glipizide. α-glucosidase inhibitors that may be useful in treating diabetes when administered with a formulation of the invention containing PACAP 66 include acarbose, miglitol and voglibose. Insulin sensitizers that may be useful in treating diabetes when administered with the formulations of the invention containing PACAP 66 include thiazolidinediones and non-thiazolidinediones. Hepatic glucose output lowering compounds that may be useful in treating diabetes when administered with the formulations of the invention containing PACAP 66 include metformin, such as Glucophage and Glucophage XR. Insulin secretagogues that may be useful in treating diabetes when administered with the formulations of the invention containing PACAP 66 include sulfonylurea and non-sulfonylurea drugs: GLP-1, GIP, PAC/VPAC receptor agonists, secretin, nateglinide, meglitinide, repaglinide, glibenclamide, glimepiride, chlorpropamide, glipizide. GLP-1 includes derivatives of GLP-1 with longer half-lives than native GLP-1, such as, for example, fatty-acid derivatized GLP-1 and exendin. In one embodiment of the invention the formulations of the invention containing PACAP 66 are used in combination with insulin secretagogues to increase the sensitivity of pancreatic beta cells to the insulin secretagogue.

Formulations of the invention containing PACAP 66 may also be used in methods of the invention in combination with anti-obesity drugs. Anti-obesity drugs include β-3 agonists, CB-1 antagonists, appetite suppressants, such as, for example, sibutramine (Meridia), and lipase inhibitors, such as, for example, orlistat (Xenical).

Formulations of the invention containing PACAP 66 may also be used in methods of the invention in combination with drugs commonly used to treat lipid disorders in diabetic patients. Such drugs include, but are not limited to, HMG-CoA reductase inhibitors, nicotinic acid, bile acid sequestrants, and fibric acid derivatives. Formulations of the invention containing PACAP 66 may also be used in combination with anti-hypertensive drugs, such as, for example, β-blockers and ACE inhibitors.

Such co-therapies may be administered in any combination of two or more drugs (e.g., the formulations of the invention containing PACAP 66 in combination with an insulin sensitizer and an anti-obesity drug). Such co-therapies may be administered in the form of pharmaceutical compositions.

The invention may be embodied in other specific forms without departing from the spirit or essential characteristics thereof. The foregoing examples are included by way of illustration only. Accordingly, the scope of the invention is limited only by the scope of the appended claims.

1. A stabilized peptide formulation, either in a solution or in a suspension, comprising:
(a) a peptide containing at least one histidine residue;
(b) a transition metal salt; and
(c) a pharmaceutically acceptable organic solvent.

2. The formulation of claim 1, wherein said peptide is selected from the group consisting of the peptide hormone superfamily containing PACAP, PACAP-like peptides, VIP, glucagon, glucagon-like peptides, secretin, helodermin, exendin-4, and functionally equivalent variants thereof.

3. The formulation of claim 1, wherein said peptide is PACAP 66 (SEQ ID NO: 1).

4. The formulation of claim 1, wherein said histidine residue is a terminal histidine residue.

5. The formulation of claim 1, wherein said peptide is selected from the group consisting of adrenocorticotropic hormone, angiotensins, renin substrate tetradecapeptide, natriuretic peptides, gastrointestinal peptides, luteinizing hormone releasing hormone, melanocyte sitmulating hormone, and neurotensin, and parathyroid hormone.

6. The formulation of claim 1, wherein said transition metal salt is a salt of a transition metal selected from the group consisting of zinc, copper, iron, manganese, nickel and cobalt.

7. The formulation of claim 6, wherein said transition metal salt is a zinc salt.

8. The formulation of claim 1, wherein said organic solvent is selected from the group consisting of DMSO, 1-methyl-2-pyrrolinidone, propanol, propylene glycol, glycerol acetate, monothioglycerol, acetic acid, diethanolamine, benzyl alcohol, ethyl lactate, glycerol formal, N-methylpyrrolidone, polyethyleneglycol 400, and isopropyl myristate.

9. The formulation of claim 1, wherein said organic solvent is a mixture of two or more organic solvents selected from the group consisting of DMSO, 1-methyl-2-pyrrolinidone, propanol, propylene glycol, glycerol acetate, monothioglycerol, acetic acid, diethanolamine, benzyl alcohol, ethyl lactate, glycerol formal, N-methylpyrrolidone, polyethyleneglycol 400, and isopropyl myristate.

10. The formulation of claim 8, wherein said organic solvent is DMSO, 1-methyl-2-pyrrolinidone, or propanol.

11. A stabilized peptide formulation, either in a solution or in a suspension, comprising:
(a) PACAP 66 (SEQ ID NO: 1) and/or salts thereof;
(b) ZnCl₂; and
(c) a pharmaceutically acceptable organic solvent.

12. The stabilized peptide formulation of claim 11, wherein said organic solvent is selected from the group consisting of DMSO, 1-methyl-2-pyrrolinidone, propanol, propylene glycol, glycerol acetate, monothioglycerol, acetic acid, diethanolamine, benzyl alcohol, ethyl lactate, glycerol formal, N-methylpyrrolidone, polyethyleneglycol 400, and isopropyl myristate.

13. The stabilized peptide formulation of claim 12, wherein said organic solvent is DMSO, 1-methyl-2-pyrrolinidone or propanol.

14. The formulation of claim 11, wherein said organic solvent is a mixture of two or more organic solvents selected from the group consisting of DMSO, 1-methyl-2-pyrrolinidone, propanol, propylene glycol, glycerol acetate, monothioglycerol, acetic acid, diethanolamine, benzyl alcohol, ethyl lactate, glycerol formal, N-methylpyrrolidone, polyethyeneglycol 400, and isopropyl myristate.

15. The formulation of claim 11, wherein said ZnCl₂ is at a ZnCl₂:peptide molar ratio of above 0.1 in said organic solvent.

16. The formulation of claim 11, wherein said PACAP 66 and/or salts thereof are at a concentration of above 0.1 mg/mL of said organic solvent.

17. A stabilized peptide formulation, comprising a dried mixture of an acid and a peptide containing at least one asparagine residue.

18. The formulation of claim 17, wherein said peptide is PACAP 66 (SEQ ID NO: 1).

19. The formulation of claim 17, wherein said acid is an inorganic acid.

20. The formulation of claim 19, wherein said inorganic acid is selected from HCl and H₃PO₄.

21. The formulation of claim 17, wherein said acid is TFA.

22. The formulation of claim 17, wherein said formulation is freeze-dried or spray-dried.

23. The formulation of claim 17, further comprising a transition metal salt.

24. The formulation of claim 23, wherein said transition metal salt is a salt of a transition metal selected from the group consisting of zinc, copper, iron, manganese, nickel and cobalt.

25. The formulation of claim 24, wherein said transition metal is zinc.

26. A stabilized peptide formulation, comprising a dried mixture of an acid and PACAP 66 (SEQ ID NO: 1) and/or a salt thereof.

27. The formulation of claim 26, wherein said acid is TFA.

28. The formulation of claim 26, wherein said acid is an inorganic acid.

29. The formulation of claim 28, wherein said inorganic acid is selected from HCl and H₃PO₄.

30. The formulation of claim 26, wherein a molar ratio of said acid to said PACAP 66 and/or a salt thereof is above 0.1.

31. The formulation of claim 26, further comprising a transition metal salt.

32. The formulation of claim 31, wherein said transition metal salt is a salt of a transition metal selected from the group consisting of zinc, copper, iron, manganese, nickel and cobalt.

33. The formulation of claim 32, wherein said transition metal is zinc.

34. A stabilized peptide formulation, comprising a dried mixture of a transition metal salt and a peptide containing at least one histidine residue.

35. The formulation of claim 34, further comprising a pharmaceutically acceptable organic solvent.

36. The formulation of claim 35, wherein said organic solvent is selected from the group consisting of DMSO, 1-methyl-2-pyrrolinidone, propanol, propylene glycol, glycerol acetate, monothioglycerol, acetic acid, diethanolamine, benzyl alcohol, ethyl lactate, glycerol formal, N-methylpyrrolidone, polyethyleneglycol 400, and isopropyl myristate.

37. The formulation of claim 36, wherein said organic solvent is DMSO, 1-methyl-2-pyrrolinidone or propanol.

38. The formulation of claim 35, wherein said organic solvent is a mixture of two or more organic solvents selected from the group consisting of DMSO, 1-methyl-2-pyrrolinidone, propanol, propylene glycol, glycerol acetate, monothioglycerol, acetic acid, diethanolamine, benzyl alcohol, ethyl lactate, glycerol formal, N-methylpyrrolidone, polyethyleneglycol 400, and isopropyl myristate.

39. The formulation of claim 34, wherein said peptide is selected from the consisting of the peptide hormone superfamily containing PACAP, PACAP-like peptides, VIP, glucagon, glucagon-like peptides, secretin, helodermin, exendin-4, and functionally equivalent variants thereof.

40. The formulation of claim 34, wherein said peptide is PACAP 66 (SEQ ID NO: 1).

41. The formulation of claim 34, wherein said peptide is selected from the group consisting of adrenocorticotropic hormone, angiotensins, renin substrate tetradecapeptide, natriuretic peptides, gastrointestinal peptides, luteinizing hormone releasing hormone, melanocyte sitmulating hormone, and neurotensin, and parathyroid hormone.

42. The formulation of claim 34, wherein said transition metal salt is a salt of a transition metal selected from the group consisting of zinc, copper, iron, manganese, nickel and cobalt.

43. The formulation of claim 42, wherein said transition metal salt is a zinc salt.

44. A process for preparing a stabilized peptide formulation, comprising the steps of:
(a) preparing an acid solution of acid and water;
(b) cooling said acid solution to below room temperature;
(c) mixing said cooled acid solution and a peptide containing at least one asparagine residue to create a cooled mixture; and
(d) drying said cooled mixture.

45. The process of claim 44, wherein said acid is an inorganic acid.

46. The process of claim 45, wherein said inorganic acid is selected from HCl and H₃PO₄.

47. The process of claim 44, wherein said acid is TFA.

48. The process of claim 44, wherein said peptide is PACAP 66 (SEQ ID NO: 1) and/or a salt thereof.

49. The process of claim 48, wherein a molar ratio of said acid to said PACAP 66 and/or a salt thereof is above 0.1

50. The process of claim 44, wherein said drying step is freeze-drying or spray-drying.

51. The process of claim 44, further comprising adding a transition metal salt to said cooled mixture before drying said cooled mixture.

52. The process of claim 51, wherein said transition metal salt is a salt of a transition metal selected from the group consisting of zinc, copper, iron, manganese, nickel and cobalt.

53. The process of claim 52, wherein said transition metal is zinc.

54. A process for preparing a stabilized peptide formulation, comprising the steps of:
(a) mixing an aqueous solution containing a transition metal salt with a peptide containing at least one histidine residue; and
(b) drying said mixture.

55. The process of claim 54, wherein said peptide is selected from the group consisting of the peptide hormone superfamily containing PACAP, PACAP-like peptides, VIP, glucagon, glucagon-like peptides, GRF, secretin, helodermin, exendin-4, and functionally equivalent variants thereof.

56. The process of claim 54, wherein said peptide is PACAP 66 (SEQ ID NO: 1).

57. The process of claim 54, wherein said peptide is selected from the group consisting of adrenocorticotropic hormone, angiotensins, renin substrate tetradecapeptide, natriuretic peptides, gastrointestinal peptides, luteinizing hormone releasing hormone, melanocyte sitmulating hormone, and neurotensin, and parathyroid hormone.

58. The process of claim 54, wherein said transition metal salt is a salt of a transition metal selected from the group consisting of zinc, copper, iron, manganese, nickel and cobalt.

59. The process of claim 58, wherein said transition metal salt is a zinc salt.

60. The process of claim 54, further comprising the step of adding a pharmaceutically acceptable organic solvent to said dried mixture.

61. The process of claim 60, wherein said organic solvent is selected from the group consisting of DMSO, 1-methyl-2-pyrrolinidone, propanol, propylene glycol, glycerol acetate, monothioglycerol, acetic acid, diethanolamine, benzyl alcohol, ethyl lactate, glycerol formal, N-methylpyrrolidone, polyethyeneglycol 400, and isopropyl myristate.

62. The process of claim 60, wherein said organic solvent is a mixture of two or more organic solvents selected from the group consisting of DMSO, 1-methyl-2-pyrrolinidone, propanol, propylene glycol, glycerol acetate, monothioglycerol, acetic acid, diethanolamine, benzyl alcohol, ethyl lactate, glycerol formal, N-methylpyrrolidone, polyethyeneglycol 400, and isopropyl myristate.

63. The process of claim 61, wherein said organic solvent is DMSO, 1-methyl-2-pyrrolinidone, or propanol.

64. The process of claim 54, wherein said histidine residue is a terminal histidine residue.

65. The process of claim 54, wherein said drying step is freeze-drying or spray-drying.


SEQUENCE LISTING (1 sequence)

SEQ ID NO: 1 - protein, 31 aa, Homo sapiens
HSDAVFTDNYTRLRKQVAAKKYLQSIKNKRY